(19) United States
(12) Patent Application Publication
(10) Pub. No.: US 2013/0197657 A1
(43) Pub. Date: Aug. 1, 2013

(54) CENTRAL AIRWAY STENT

(71) Applicants: Diana ANCA, New York, NY (US); Faiz BHORA, New York, NY (US)

(72) Inventors: Diana ANCA, New York, NY (US); Faiz BHORA, New York, NY (US)

(21) Appl. No.: 13/693,755

(22) Filed: Dec. 4, 2012

Related U.S. Application Data
(60) Provisional application No. 61/568,557, filed on Dec. 8, 2011.

(51) Int. Cl. A61F 2/04

(52) U.S. Cl. CPC A61F 2/04
USPC 623/23.7

(57) ABSTRACT

The present invention relates to central airway stents, and methods and devices for deploying such stents. The central airway stents are useful for maintaining or prolonging the patency of a central airway.

### FIELD OF THE INVENTION

Devices and methods for relieving an obstruction of a central airway are provided. In particular, a stent suitable for deployment in a central airway, and methods and devices for deploying the stent are provided. Further, methods and devices for maintaining or prolonging the patency of a central airway are provided

### BACKGROUND

Lung cancer is the most common causes for obstruction of a central airway. However, other cancers and benign causes for airway obstruction also exist, such as, tracheobronchomalacia and obstructive sleep apnea. Symptoms of airway obstruction may include shortness of breath during exertion or at rest, respiratory failure, or asphyxia requiring ventilatory support. Consequently, morbidity is high among patients afflicted with a central airways obstruction in the absence of any intervention. Airway patency can be improved by surgical or endoscopic methods, thereby improving quality of life and, possibly, survival rate. Surgical resection is effective among early stage lung cancer patients but airway stenting, with or without surgical resection or other treatment, is more beneficial for patients with advanced lung cancer or having airway obstruction due to non malignant causes. A central airway stent is often used in conjunction with chemo- or radio-therapy.

Several types of central airway stents, as well as method and devices to deploy them are known, such as, those disclosed in U.S. Pat. Nos. 5,184,610; 5,480,431; 7,997,266 and U.S. Patent Application Publication No. 2003/0024534 A1. However, the marketed airway stents often present problems, such as, mucus plugging and stent migration (Razi et al., Ann. Thorac. Surg., 2010, 90:1088-93), that are inherent function of the mismatch of the structure of these stents with the normal anatomy of the central airways. Solutions to the mismatch problem have been proposed. For example, U.S. Pat. No. 5,480,431 discloses a metal alloy stent that can expand upon being exposed to an elevated temperature. Such stents, however, expand uniformly throughout their length. Thus, one end of the stent may be of suitable diameter for the anatomy, while the other end may apply too much pressure on the airway wall, which can result in pressure necrosis and an esophageal fistula.

### SUMMARY

In one aspect of the invention, stents suitable for deployment in a central airway are provided. In another aspect, deployment devices comprising a central airway stent of the invention and means for deploying the stent are provided. In another aspect, the present invention provides methods for inserting a central airway stent in a patient. In yet another aspect, the present invention provides methods for repositioning a central airway stent of the invention in a patient.

In one embodiment, a central airway stent comprises a tapered tube having a proximal open end and a distal open end, wherein the proximal open end has a diameter greater than the distal open end.

In certain embodiments of the invention, the central airway stent comprises an elongation portion, which may be, for example, a telescoped portion or a pleated portion.

In another embodiment, an accordion type central airway stent is disclosed comprising a tube having:

(a) a proximal open end,

(b) a distal open end, and

(c) a circumferential pleated portion between the proximal open end and the distal open end.

In yet another embodiment, a telescoped central airway stent is disclosed comprising a tube having:

(a) a proximal open end,

(b) a distal open end,

wherein a portion of the tube lying between the proximal open end and the distal open end is invaginated toward the proximal open end.

In a further embodiment, a telescoped central airway stent is disclosed comprising:

(a) a proximal tube, and

(b) a distal tube having a diameter less than the diameter of the proximal tube,

wherein at least a portion of the distal tube resides within the proximal tube.

In a further embodiment, a telescoped central airway stent is disclosed comprising:

(a) a proximal tube, and

(b) a distal tube having a diameter less than the diameter of the proximal tube,

wherein the distal tube may be completely separated from the proximal tube at the time of deployment to form a modular stent system.

In another embodiment, the central airway stent of the invention further comprises two narrower tubes lodged within the distal tube, or distal end of the tube, wherein at least a major portion of each of the narrower tubes resides within the distal tube or distal end of the tube before deployment.

In some embodiments, the two narrower tubes may be of differing diameters so as to be placed concentrically inside the distal tube or distal end of the tube, or they may be placed adjacent to each other inside the distal tube or distal end of the tube.

In other embodiments, the central airway stent of the invention may have part of the distal tube or distal end of the tube branch into two narrower tubes that are suitably flexible to lie adjacent to each other in substantially parallel position when lodged in a deployment device.

In yet other embodiments, the central airway stent of the invention may have an additional tube, connected to the distal tube or distal end of the tube, having a diameter less than the diameter of the distal tube or distal end of the tube, wherein the additional tube branches into two narrower tubes that are flexible enough to lie adjacent to each other in a substantially parallel position while lodged in the distal tube or distal end of the tube before deployment.

In another embodiment, the present invention provides a method for inserting a central airway stent in a patient comprising:

(a) endotracheally inserting a deployment device comprising the central airway stent of the invention into the central airway of a patient; and

(b) deploying the central airway stent.

In an embodiment, a method for repositioning a central airway stent having a circumferential pleated portion between the proximal open end and the distal open end comprises pulling the proximal open end or the distal open end sufficiently to at least partially straighten at least one pleat of a stent.

In another embodiment, a method for repositioning a telescoped central airway stent comprises pulling the proximal open end or the distal open end sufficiently to at least partially decrease the length of the invaginated portion.

In yet another embodiment, a method for repositioning the central airway stent comprises reducing the length of the portion of the distal tube that resides within the proximal tube to increase the length of the distal tube that is not within the proximal tube.

In yet another embodiment, a method for deploying the central airway stent comprises pushing out the two narrower tubes that reside within the distal tube or the distal end of the tube to form a Y shaped stent deployed in the trachea with suitable branching at the carina into the right and left main bronchi.

The narrower tubes may be pushed out together at or close to the carina after the deployment of the main tube(s) if they are lying adjacent to each other in the distal tube or distal end of the tube. The narrower tubes may be pushed out consecutively after the deployment of the main tubes if they are concentric to each other in the distal tube or distal end of the tube, such that one narrow tube is deployed in each main bronchus to form a modular system.

The narrower tubes may be pushed out together at a conveniently short distance from the carina during deployment of the main tubes, if the narrower tubes are extensions of the distal tube or distal end of the tube, lying adjacent to each other in a deployment device, such that they are sufficiently separated to be guided conveniently into the two bronchi without causing puncture or abrasion to the trachea that would result from moving a fully formed Y or T shaped stent across the length of the trachea.

The stents may have proximal and distal radio-opaque markers that help with stent deployment.

### BRIEF DESCRIPTION OF THE DRAWINGS

FIG. 1 depicts a conical stent, and having a distal open end 1, a proximal open end 2 and a tapered tube 3 having a metallic mesh structure 4 and a lining 5.

FIG. 2 depicts an accordion stent having a tube 6 with a distal open end 1, a proximal open end 2 and an elongation portion 7 having pleats 8.

FIG. 3 depicts a telescoped stent and its cross section, having a distal portion 9, a proximal portion 10, a distal open end 1, a proximal open end 2, and an elongation portion 7 having an invaginated portion 11.

FIG. 4 depicts a telescoped stent and its cross sections having a distal tube 12, a proximal tube 13, and an elongation portion 7.

FIG. 5 depicts a telescoped stent that forms a modular system having a distal tube 12, and a proximal tube 13.

FIG. 6 depicts a cross section of telescoped stent inside a deployment device 17, the stent having a proximal tube 13, a distal bifurcated tube 14 branched into narrower tubes 15 and 16, and an elongation portion 7.

FIG. 7 depicts a cross section of telescoped stent having a distal tube 12, a proximal tube 13, and an elongation portion 7, and concentric tapered narrower tubes 18 and 19.

FIG. 8 depicts a cross section of telescoped stent having a distal portion 9, a proximal portion 10, an elongation portion 7 with invaginated portion 11, and an additional distal bifurcated tube 14 branched into narrower tubes 15 and 16.

### DETAILED DESCRIPTION

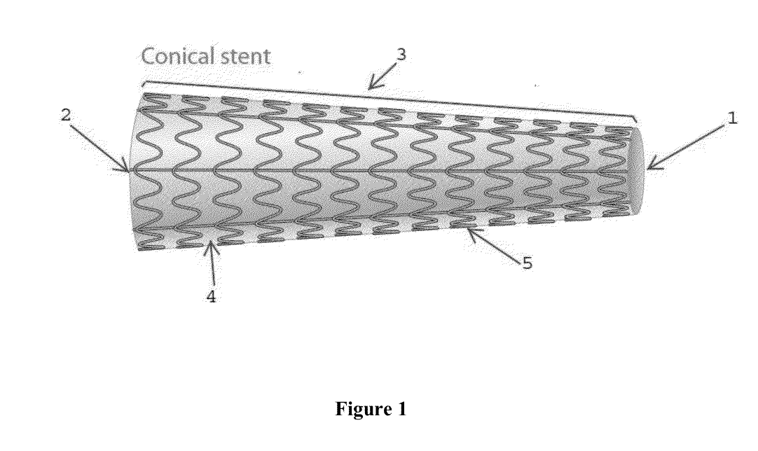

FIGS. 1 to 8 illustrate certain embodiments of the invention. FIG. 1 illustrates a central airway stent of conical shape having a tapered tube 3 with proximal open end 2 having a diameter greater than the distal open end 1 of the tube. The stent has a metallic mesh structure 5 with a lining 4. In some embodiments, the lining may be of synthetic or natural polymeric material. In some embodiments, the metallic mesh may be of shape-memory alloy.

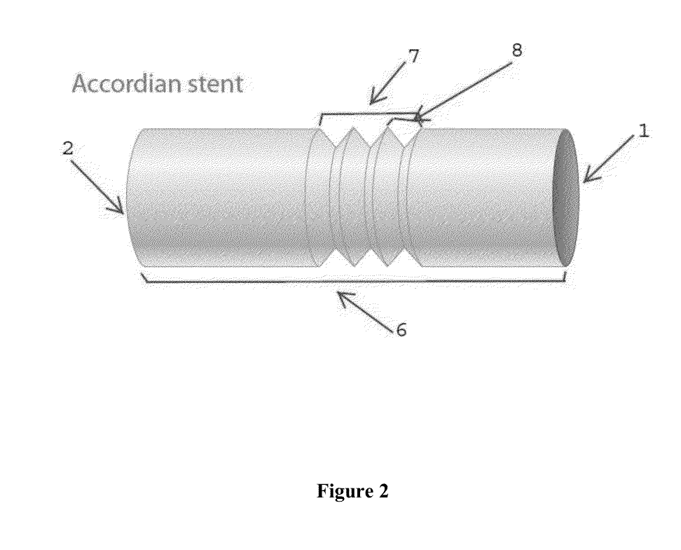

FIG. 2 illustrates a central airway stent of an accordion type structure having a tube 6 with a distal open end 1, a proximal open end 2 and circumferential pleated elongation portion 7 having three pleats 8. In other embodiments, the accordion type stent may have one or more pleats, such as, 2 to 4 pleats. Each pleat may have an effective length of about 0.2 cm to about 1 cm when fully straightened. In some embodiments, the accordion type stent may have proximal open end 2 having a diameter greater than the distal open end 1. The central airway stent may be repositioned by pulling the proximal open end 2 or the distal open end 1 sufficiently to at least partially straighten one or more of the pleats 8.

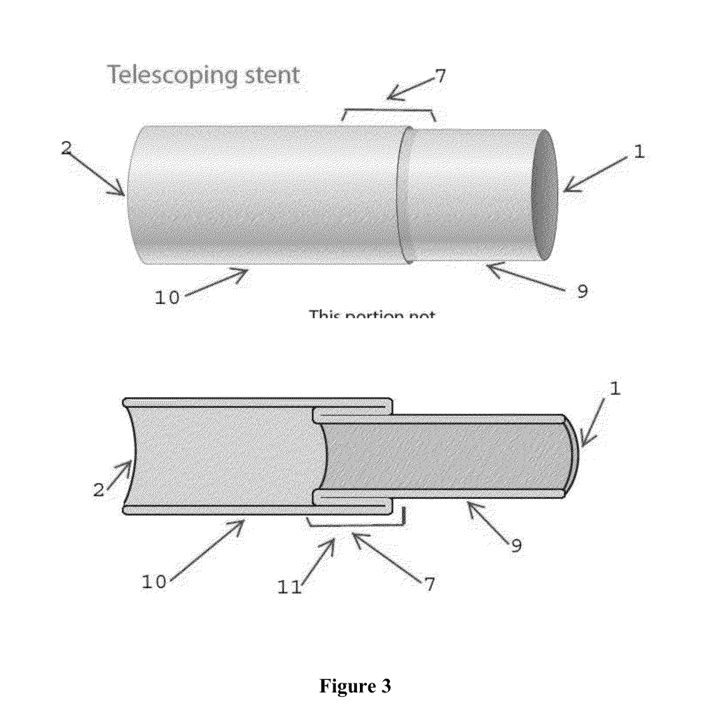

FIG. 3 illustrates a central airway stent of a telescopic type structure, and its cross section, having a distal portion 9, a proximal portion 10, wherein a portion 11 of the tube lying between the proximal open end 2 and the distal open end 1 is invaginated toward the proximal open end 2. The central airway stent may be repositioned by pulling the proximal open end 2 or the distal open end 1 sufficiently to at least partially decrease the length of the invaginated portion 11 which constitutes the elongation portion 7 for the stent of FIG. 3. In some embodiments, the telescopic type stent may have proximal open end 2 having a diameter greater than the distal open end 1. The maximum length of the invaginated portion 11 of the telescoped stent may be from about 0.5 cm to about 3 cm, such as about 2 cm.

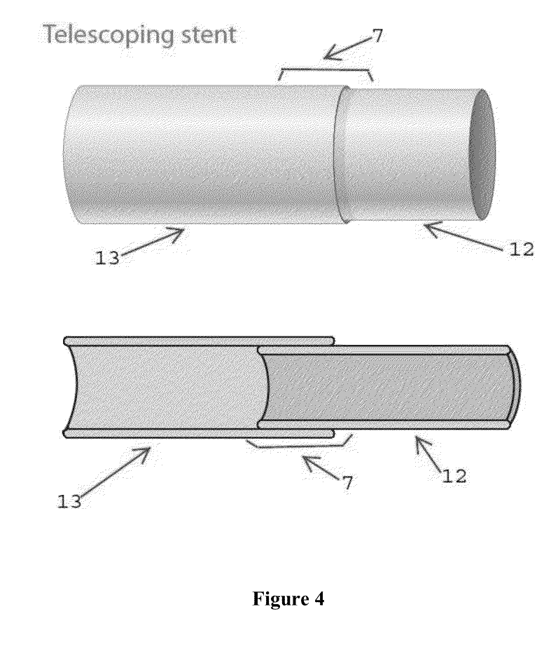

FIG. 4 illustrates a central airway stent of a telescopic type structure, and its cross section, having a distal tube 12 having a diameter less than the diameter of the a proximal tube 13, wherein at least a portion of the distal tube 13 resides within the proximal tube 12. The central airway stent may be repositioned by reducing the length of this elongation portion 7 of the distal tube that resides within the proximal tube to increase the length of the distal tube that is not within the proximal tube. In some embodiments, the telescopic stent may have tapered distal tube 13 residing within a tapered proximal tube 12.

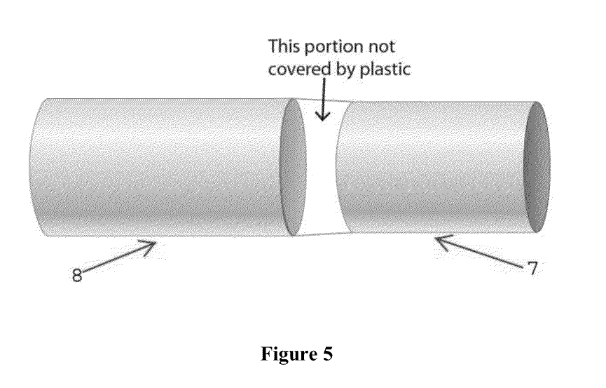

FIG. 5 illustrates a central airway stent of a telescopic type structure, and its cross section, having a distal tube 12 having a diameter less than the diameter of the a proximal tube 13, wherein the tubes are completely separated from each other to provide a modular stent system. At least a portion of the distal tube 13 can suitably reside within the proximal tube 12 at the time of deployment.

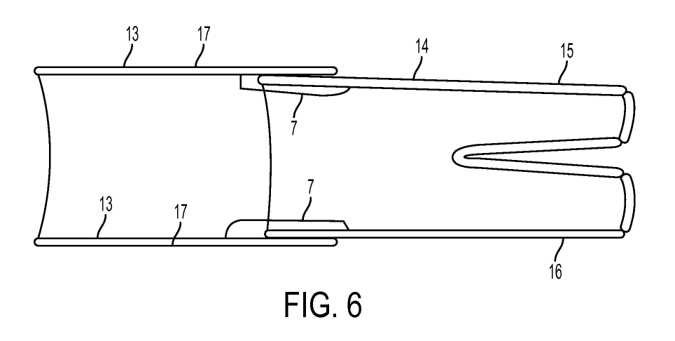

FIG. 6 illustrates a cross section of a central airway stent of Y type structure, having an elongation portion 7, a distal tube 14 having a diameter less than the diameter of the a proximal tube 13, wherein the distal bifurcated tube 14 branches into narrower tubes 15 and 16 that are suitably flexible to lie adjacent to each other in substantially parallel position while lodged in the deployment device 17. The portion of the tube 14 and narrower tubes 15 and 16 where the bifurcation or branching occurs may be made of suitable synthetic or natural polymeric material to impart desired flexibility while other parts may be of more inflexible material so as to function effectively and maintain airflow.

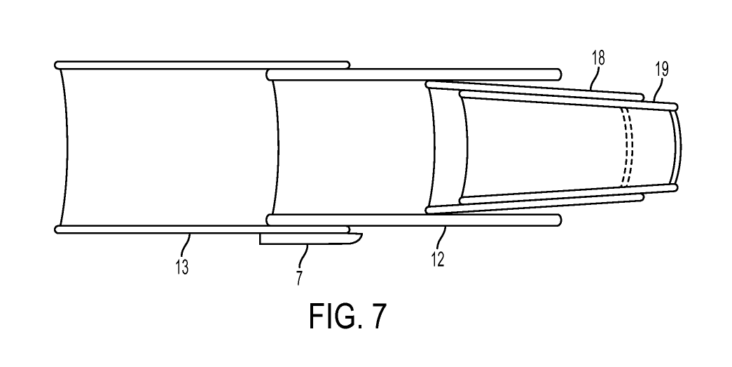

FIG. 7 illustrates a cross section of a central airway stent of modular Y type structure, having a distal tube 12, a proximal tube 13, and an elongation portion 7 wherein at least a portion of the distal tube 13 resides within the proximal tube 12, and concentric tapered narrower tubes 18 and 19. The narrower tubes 18 and 19 may be pushed out consecutively after the deployment of the main tubes, such that one narrow tube is deployed in each main bronchus to form a modular system, the tube 18 with the larger diameter being deployed in the left bronchus.

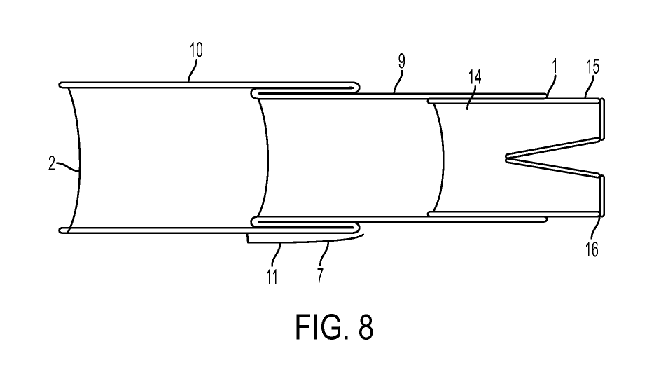

FIG. 8 illustrates a cross section of a central airway stent of Y type structure, having a distal portion 9, a proximal portion 10, an elongation portion 7 with invaginated portion 11, and an additional distal bifurcated tube 14 branched into narrower tubes 15 and 16 that are flexible enough to lie adjacent to each other in a substantially parallel position while lodged in the distal tube or distal end of the tube before deployment. The bifurcated tube 14 has a diameter less than the diameter of the distal portion 10. The portion of the tube 14 and narrower tubes 15 and 16 where the bifurcation or branching occurs may be made of suitable synthetic or natural polymeric material to impart desired flexibility while other parts may be of more inflexible material so as to function effectively and maintain airflow.

The central airway stents of the invention may be tapered or straight. The central airway stents may be circular, oval or squarish-oval in cross section. The central airway stents may be conical or of other suitable shapes.

The narrower tubes 15 and 16 may be of identical size or one of the narrower tubes may be narrower or shorter than the other.

The proximal open end 2 may have a diameter of about 16 mm to about 22 mm, such as, a diameter of about 20 mm, suitable for deployment in the Trachea. The proximal open end 2 may have a diameter of about 10 mm to about 16 mm, such as, a diameter of about 14 mm, suitable for deployment in the main bronchi. The distal open end 1 may have a diameter of about 14 mm to about 22 mm, such as, a diameter of about 18 mm for deployment in the Trachea. The distal open end 1 may have a diameter of about 8 mm to about 14 mm, such as, a diameter of about 10 mm, suitable for deployment in the main bronchi. The stent may have a length of from about 2 cm to about 10 cm, such as 2 cm, 3 cm, 4 cm, 5 cm, 6 cm, 8 cm, 9 cm, or 10 cm.

The length of distal tube 12, proximal tube 13 and narrower tubes 15 and 16, or 18 and 19 are proportionate to the overall length of the stent and may be from about 0.5 cm to about 6 cm.

Similarly, the maximum length of the portion of the distal tube that lies inside the proximal tube of the telescoped stent may be from about 0.5 cm to about 3 cm, such as about 2 cm.

The edges of the proximal open end 2 and the distal open end 1 of the tubes may be beveled, rounded or wedged so that positioning and repositioning in the trachea can be carried out smoothly without causing abrasions.

The central airway stent may be made of synthetic, natural, or biodegradable material or made of metal, including shape-memory alloys. The construction of the tube may be covered, such as made from plain sheets, or open, such as made of meshed or woven metal wires, or polymeric threads. Examples of suitable materials include silicon rubber, such as Dow Corning 3110 RTV; polyurethane; polyethylene terephthalate, such as Dacron®, Polytetrafluoroethylene (PTFE), such as teflon®; nickel titanium alloys, such as nitinol; and other metal alloys, such as FePt, FePd, FeNiCoTi, FeNiC, FeMnSi or FeMnSiCrNi. The materials may be coated or otherwise infused with a therapeutic medicament that provides local treatment to the central airway. Non-limiting examples of therapeutic medicaments include chemotherapeutic agents and anti-inflammatory agents. A central airway stent according to the present invention may also comprise agents for radiation therapy and delivery.

According to the present invention, a central airway stent may comprise a biodegradable or bioabsorbable material. Such a stent is advantageous for patients who require a stent for a period of weeks or months because, e.g., their central airway tumor has been responsive to treatment. Non-limiting examples of suitable biodegradable or bioabsorbable materials include, Polycaprolactone; Poly(L-lactide), Poly(DL-lactide); Polyglycolide; Poly(L-Lactide-co-D,L-Lactide) 70:30 Poly(L-Lactide-co-D,L-Lactide); 95:5 Poly(DL-lactide-co-glycolide); 90:10 Poly(DL-lactide-co-glycolide); 85:15 Poly(DL-lactide-co-glycolide); 75:25 Poly(DL-lactide-co-glycolide); 50:50 Poly(DL-lactide-co-glycolide); 90:10 Poly(DL-lactide-co-caprolactone); 75:25 Poly(DL-lactide-co-caprolactone); 50:50 Poly(DL-lactide-co-caprolactone); Polydioxanone; Polyesteramides; Copolyoxalates; Polycarbonates and Poly(glutamic-co-leucine). Polydioxanone; poly-96L/4D-lactic acid [PLA], and Type I collagen from bovine sources.

Suitable deployment devices include conventional deployment devices known in the art, which have been loaded with a central airway stent according to the present invention. Examples of such suitable deployment devices are the AERO® Tracheal Over-the-Wire Delivery Catheter and AERO® Bronchial Over-the-Wire Delivery Catheter from Merit medical Endotek, and other deployment devices disclosed in U.S. Pat. No. 6,409,750, and U.S. Patent Application Publication No. 2009/0319021 A1.

Suitable deployment procedures include conventional methods known in the art, such as those disclosed in U.S. Pat. No. 6,409,750, and U.S. Patent Application Publication No. 2009/0319021 A1. For deployment, the patient is usually positioned in a supine position. Access to the airway is obtained either through an endotracheal tube, rigid bronchoscope, laryngeal mask, suspension device, or other such airway designed to provide airway access. General anesthesia or other anesthesia with sedation can be used for stent deployment as suitable to the clinical situation. Stents according to the invention are preloaded on the delivery system, which includes a relatively flexible introduction catheter. Once appropriate position of the stent/delivery system has been confirmed by bronchoscopic evaluation and fluoroscopy, the sheath or thread that constrains the stent is slowly released, and the stent gradually expands within the airway, conforming to the walls of the bronchus or trachea. The radioopaque markers assist with correct positioning of the stent within the airway.

Examples of Y tubes, where a distal tube or distal end of a tube branches into two narrower tubes that are flexible enough to lie adjacent to each other in substantially parallel position, include suitable tubes disclosed in U.S. Pat. No. 5,480,431, and U.S. Patent Application Publication No. 2009/0319021 A1.

Specific embodiments has been described above to illustrate the invention, and are not intended to be restrictive. Modifications and equivalents will be apparent to those skilled in the art and are included within the scope of the invention. Patents and publications cited in this application are hereby incorporated by reference in their entirety.

1-31. (canceled)

32. A telescoped central airway stent comprising a tube having:
a. a proximal open end,
b. a distal open end,
wherein a portion of the tube lying between the proximal open end and the distal open end is invaginated toward the proximal open end.

33. The central airway stent of claim 32 wherein the stent is tapered, and the proximal open end has a diameter greater than the distal open end.

34. The central airway stent of claim 32 wherein the proximal open end has a diameter of about 10 mm to about 22 mm.

35. The central airway stent of claim 34 wherein the proximal open end has a diameter of about 14 mm.

36. The central airway stent of claim 32 wherein the proximal open end has a diameter of about 10 mm to about 16 mm.

37. The central airway stent of claim 36 wherein the proximal open end has a diameter of about 12 mm.

38. The central airway stent of claim 32 wherein the distal open end has a diameter of about 14 mm to about 22 mm

39. The central airway stent of claim 38 wherein the distal open end has a diameter of about 16 mm.

40. The central airway stent of claim 32 wherein the distal open end has a diameter of about 8 mm to about 14 mm.

41. The central airway stent of claim 40 wherein the distal open end has a diameter of about 10 mm.

42. The central airway stent of claim 32 wherein the edges of the proximal open end and the distal open end are beveled.

43. The central airway stent of claim 32 wherein the stent has a length of about 2 cm to about 10 cm.

44. The central airway stent of claim 43 wherein the stent has a length of 2 cm, 3 cm, 4 cm, 5 cm, 6 cm, 8 cm, 9 cm, or 10 cm.

45. A deployment device comprising the central airway stent of claim 32 and a means for deploying the central airway stent.

46. A method for inserting a central airway stent in a patient comprising:
a. endotracheally inserting a deployment device comprising the central airway stent of claim 32 into the central airway of a patient; and
b. deploying the central airway stent.

47. A method for repositioning the central airway stent of claim 32 comprising pulling the proximal open end or the distal open end sufficiently to at least partially decrease the length of the invaginated portion.

48-59. (canceled)

60. The telescoped central airway stent of claim 37 wherein the stent has a length of 2 cm, 3 cm, 4 cm, 5 cm, 6 cm, 8 cm, 9 cm, or 10 cm.

61-63. (canceled)

64. The central airway stent of claim 32, further comprising two smaller tubes lodged within the distal end of the tube, wherein at least a major portion of the smaller tubes resides within the distal end of the tube before deployment.

65-67. (canceled)

68. The central airway stent of claim 32, wherein part of the distal end of the tube is branched into two smaller tubes that are flexible enough to lie adjacent to each other in substantially parallel position when lodged in a deployment device.

69. (canceled)

70. The central airway stent of claim 32, comprising an additional tube connected to the distal tube or distal end of the tube, having a diameter less than the diameter of the distal tube or distal end of the tube, wherein the additional tube branches into two smaller tubes that are flexible enough to lie adjacent to each other in a substantially parallel position while lodged in a deployment device.

71-73. (canceled)

74. A central airway stent comprising two smaller tubes lodged within a larger tube wherein the larger tube and/or at least one of the smaller tubes is configured according to claim 32.
